(19) United States
(12) Patent Application Publication
(10) Pub. No.: US 2020/0376176 A1
(43) Pub. Date: Dec. 3, 2020

(54) Surgical Suction Device

(71) Applicant: Damon A. Alavekios, Monterey, CA (US)

(72) Inventor: Damon A. Alavekios, Monterey, CA (US)

(21) Appl. No.: 15/931,757

(22) Filed: May 14, 2020

Related U.S. Application Data
(60) Provisional application No. 62/854,639, filed on May 30, 2019.

(51) Int. Cl. A61M 1/00

(52) U.S. Cl. CPC A61M 1/0039; A61M 1/009; A61M 2205/8206; A61M 2205/07; A61M 1/0056

(57) ABSTRACT

The present device includes a tube-free, cordless, battery-powered suction device. Activation of the device can be intermittent via depression of a trigger that allows for instantaneous awareness of fluid volume loss. In an example, the present device is a lightweight, suction hand unit including an indwelling impeller motor, removable and rechargeable lithium ion battery pack that is hermetically sealed, removable fluid reservoirs, a variety of replaceable nozzle tips, and an ULPA filter.

### CROSS-REFERENCE TO RELATED APPLICATIONS

This application incorporates by reference and claims the benefit of priority to U.S. Provisional Application 62/854,639 filed on May 30, 2019.

### BACKGROUND OF THE INVENTION

The present subject matter relates generally to a tube-free, cordless, battery powered, handheld intermittent surgical suction device.

Medical personnel including nurses, doctors, paramedics, and medical technicians are often required to quickly remove unwanted liquid and/or gas from patients including vomit, secretions, foreign material, and blood from a patient's airway, wound, blood vessels, surgical field, etc. Typically, suction is provided through a suction catheter or wand inserted into the airway, wherein the unwanted material is suctioned through catheter and, optionally, into a container.

Conventional suction devices are typically not portable and, instead, used in a permanent location to provide a constant source of suction force. For example, hospital facilities can include wall-mounted suction fitting to which a medical personnel can attach suction devices, such as a suction catheter, to the wall unit to provide suction. As a result, the conventional devices require a storing space in the hospital and require patients to come to the unit (instead of the unit coming to the patient no matter where the patient is located).

The conventional permanent suction units are also not energy efficient because during a medical procedure, the suction may only be needed intermittently, however, because of the device design, the suction will remain on during the entire medical procedure (e.g., instead of turning on and off the machine, which would take up valuable time). Conventional devices also require valuable time while the patient is under anesthesia to set up, which results in increased cost for hospitals and extending the time under anesthetic for patient. Therefore, the conventional devices create large recurring costs for the healthcare system and accrues a large volume of medical waste.

The conventional devices also create a variety of hazards. For example, the conventional devices typically include power plug-ins and cords, which add to the complexity of the device and results in a tripping hazard for the medical personnel. Moreover, the conventional surgical suction devices typically use tubing that compromises sterility, mobility, endangers staff as a potential hazard (i.e. tripping over), and endangers patient, by compromising sterile field since much of the tubing is non-sterile. Further, suction provided by conventional devices can be quite loud and, as a result, can be a source of potential medical errors in interfering with communication.

The conventional industrial wall-mounted suction units are not suitable for field operations, such as for emergency medical providers or paramedics away from the hospital. Accordingly, there is a need for a portable, cordless, handheld suction device.

### BRIEF SUMMARY OF THE INVENTION

The present disclosure provides a handheld surgical suction device. Various examples of the device are provided herein.

The present disclosure provides a tube-free, cordless, battery-powered suction device. Activation of the device can be intermittent via depression of a trigger that allows for instantaneous awareness of fluid volume loss.

In an example, the present device is a lightweight, suction hand unit including an indwelling impeller motor, removable and rechargeable lithium ion battery pack that is hermetically sealed, removable fluid reservoirs, a variety of replaceable nozzle tips, and an ULPA filter.

The present device is safer to patients and medical staff by eliminating suction tubing and power cords. As a result of not having any suction tubing, the device reduces or eliminates sterility hazards resulting from the suction tubing. The device also eliminates safety hazards (e.g., tripping hazards) from the use of cords and tubes.

Advantages of the present device include minimal recurring costs, easier to operate than conventional devices, and requires minimal training, and minimal maintenance. As a result, the present device shortens patient time under anesthesia.

Another advantage of the present device is providing a tubing-free suction for removal of surgical fluids in one handheld unit.

Another advantage of the present device is quantifying and/or visualizing the exact volume of blood or fluid lost via suction.

A further advantage of the present device is providing a reduction of intraoperative background noise as compared to conventional suction devices.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a schematic of an example of the present device including separate fluid and gas suction intakes.

FIG. 2 is a schematic of an example of the present device including combined fluid and gas suction intake in fluid communication with the fluid reservoir and air filter, wherein the suction is provided by a turbine in series with the fluid reservoir.

FIG. 3 is a schematic of an example of the present device including combined fluid and gas suction intake in fluid communication with the fluid reservoir and air filter, wherein the suction is provided by a pump in series with the fluid reservoir.

FIG. 4 is a schematic of an example of the present device including combined fluid and gas suction intake in fluid communication with the fluid reservoir and air filter, wherein the suction is provided by a pump in series with the intake, before the fluid reservoir.

FIG. 5 is a schematic of an example of the present device including a separate compressed air intake, wherein the venture valve is shared between the compressed air intake and the gas and fluid intake.

FIGS. 6A-6B are perspective views of examples of the present device including the handheld configuration with the fluid reservoir positioned in front of the handle.

FIGS. 7A-7B are perspective views of examples of the present device including the handheld configuration with the fluid reservoir positioned as an extension of the handle.

FIGS. 8A-8B are perspective views of examples of the present device including a handheld configuration wherein the suction tube is connected to an external pump or motor.

FIGS. 9A-9B are perspective and exploded views, respectively, of an example of the present device.

### DETAILED DESCRIPTION OF THE INVENTION

The present suction device 10 can be portable and include a hand piece 12 including a barrel portion 14 and grip portion 16 (e.g., handle). The grip portion 16 is configured for a user to grasp the device 10. The grip portion 16 can contain a housing 18 to receive a battery 20 (e.g., a battery pack magazine). The device 10 can also include a motor connected to the battery 20, wherein the motor is configured to create the vacuum and draw in fluid and/or gas through the barrel portion 14 into a fluid reservoir 30. The device 10 can include an impeller connected to the motor that can be positioned above the hand piece 12 to create the vacuum for suction.

The fluid reservoir 30 is a container in fluid communication to the suction tube 34, wherein the reservoir 30 can receive and collect the suctioned fluid and/or gas. The fluid reservoir 30 can be positioned below the barrel portion 14 and in front of the grip portion 16. Alternatively, the fluid reservoir 30 can be positioned behind the grip portion 16.

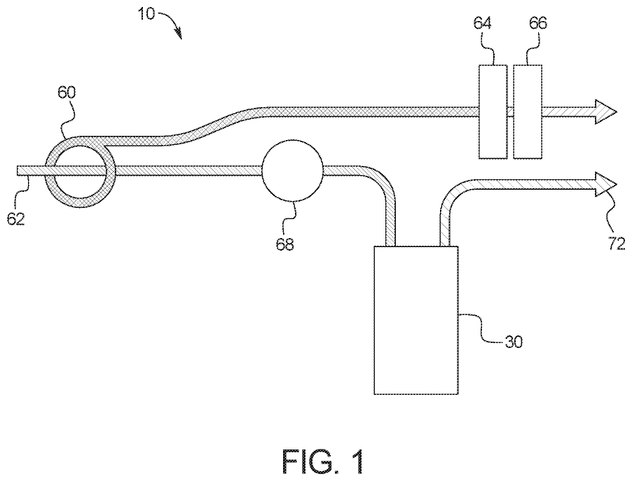

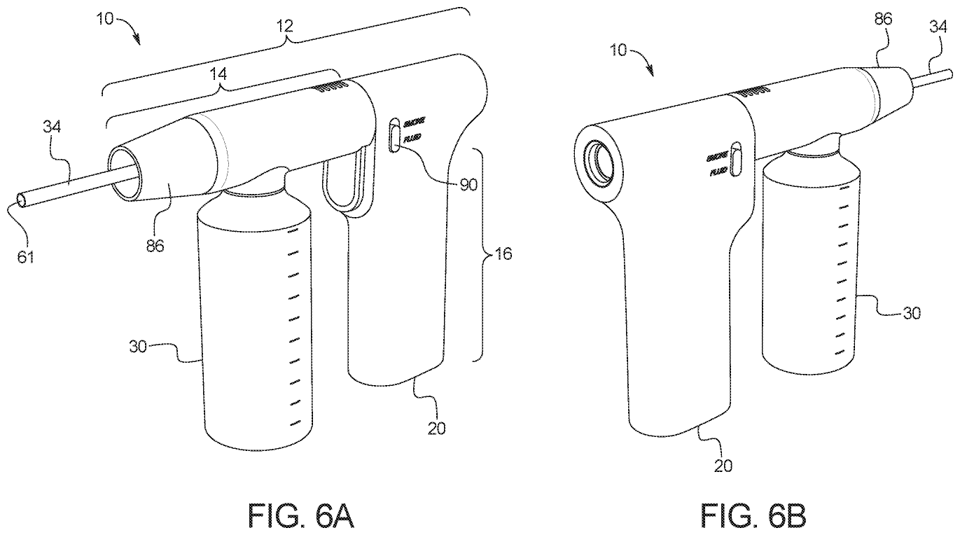

The present device can be configured in a variety of ways, including, but not limited to, the illustrated configurations in the figures. FIGS. 1-5 illustrate schematics for a variety of mechanical configurations. FIGS. 6A-9B illustrate a variety of different structural configurations.

As shown in FIG. 1, the device 10 can include two separate intake portions including a gas intake 60 and a fluid intake 62, wherein the gas intake 60 can be in communication with a HEPA filter 64 and/or turbine fan 66. The fluid intake 62 can be in communication with a pump 68 (e.g., peristaltic pump), a fluid reservoir 30, and a tank pressure vent 72. Advantages of the separate fluid and gas intakes include having specialized pumps, one for each medium, reducing consumption of power when only one medium needs to be suctioned, and quieting suction noises.

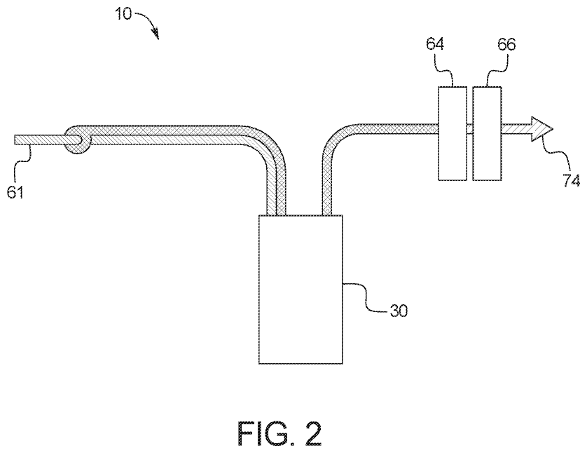

FIG. 2 illustrates a combined fluid and gas extraction device. Both the gas and fluid are combined in one intake 61 that is in fluid communication with the fluid reservoir 30, a HEPA filter 64, and turbine 66 to produce a filtered air output 74. The combination of gas and liquid intake simplify and streamline the components of the device.

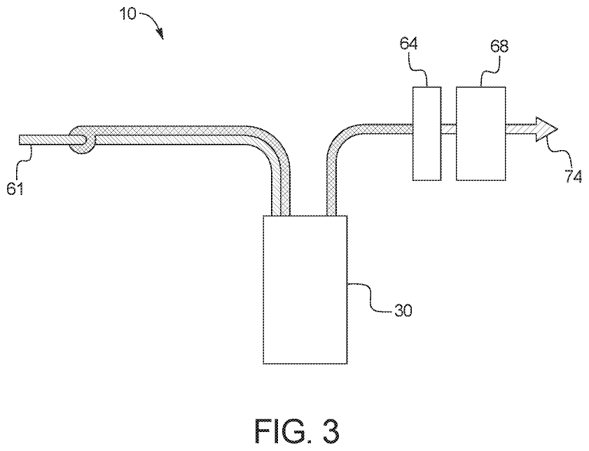

FIG. 3 is an example of a combined gas and fluid intake 61 in communication with the fluid reservoir 30, the HEPA filter 64, and a piston pump 68 positioned after the fluid reservoir 30 to create an output of filtered air 74.

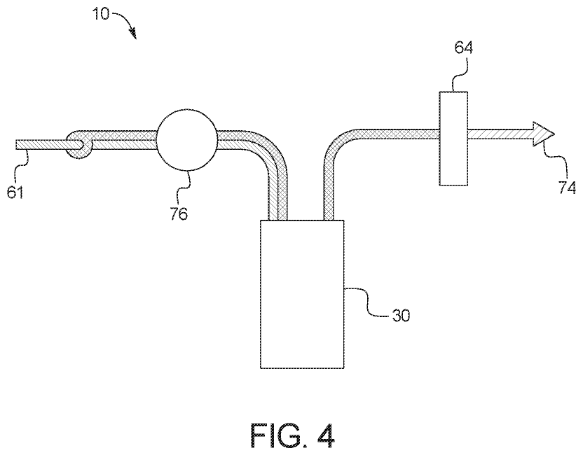

FIG. 4 is another example of combined gas and fluid intake 61 in communication with the fluid reservoir 30 and the HEPA filter 64 positioned after the reservoir 30 to create an output of filtered air 74, wherein a peristaltic pump 76 is positioned before the fluid reservoir 30. The device 10 may include a small turbine fan to suction air.

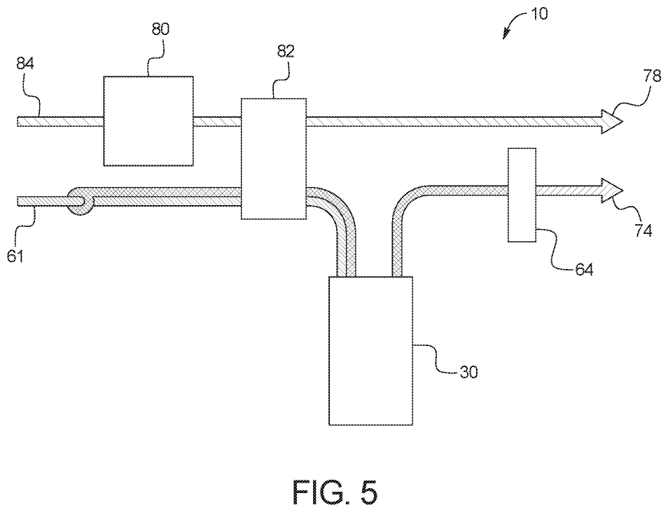

FIG. 5 is an example of the device 10 having combined gas and liquid extraction including a compressed air intake 84. In the example, the device 10 includes a single gas and fluid intake 61 in communication with a venture valve 82, a fluid reservoir 30, and an air filter 64. In addition, the device 10 includes a compressed air intake 84 in communication with the venture valve 82 and air compressor 80. Such configuration isolates the motor from the fluid path. Advantages of a configuration including the venture valve 82 include increased quietness, high suction pressure, separate motor and fluid path.

FIG. 6A illustrates a device 10 having a single fluid/gas intake tube 64, a fluid reservoir 30, and a handle portion 16 containing the power source (e.g., battery 20).

As shown in FIG. 6B, the device 10 can include an optional bypass port to bypass canister and connect up to wall mounted or floor mounted suction for high volume cases or rapid increase in blood loss. The device 10 can include a sliding indwelling suction tip 86 to allow for a user to change the length of the suction tube 34. The sliding indwelling suction tip 86 can include laser lines for measurement purposes. The device can include a suction selector 90 on the handle portion 16 to select from gas or liquid suction.

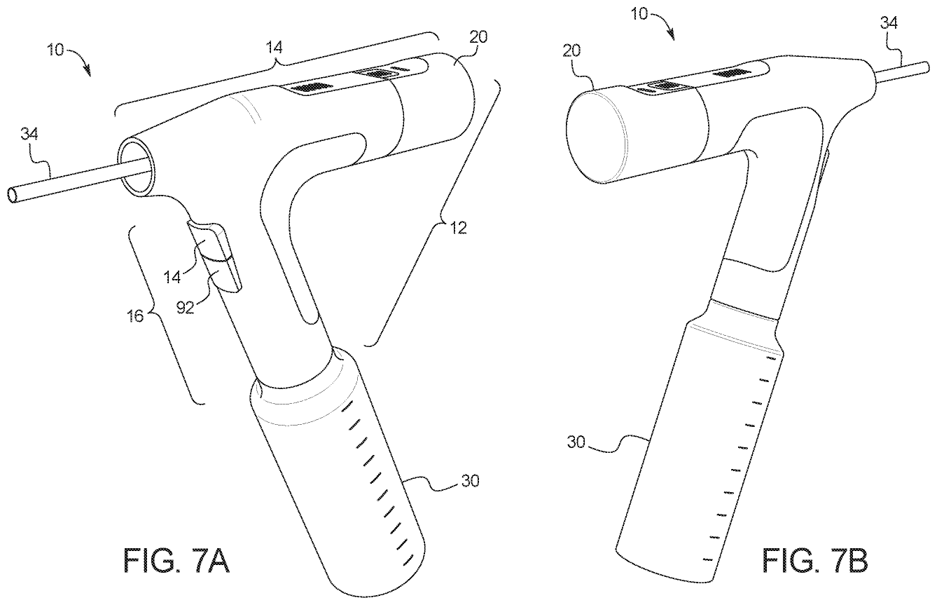

FIGS. 7A-7B illustrate an example of the device 10 wherein the fluid reservoir 30 is contained within or below the handle portion 16 of the device 10. In such example, the battery pack 20 is connected to the back end of the pistol portion 14. The device 10 can include a dual trigger 92 on the handle portion 16 to select from gas or liquid suction.

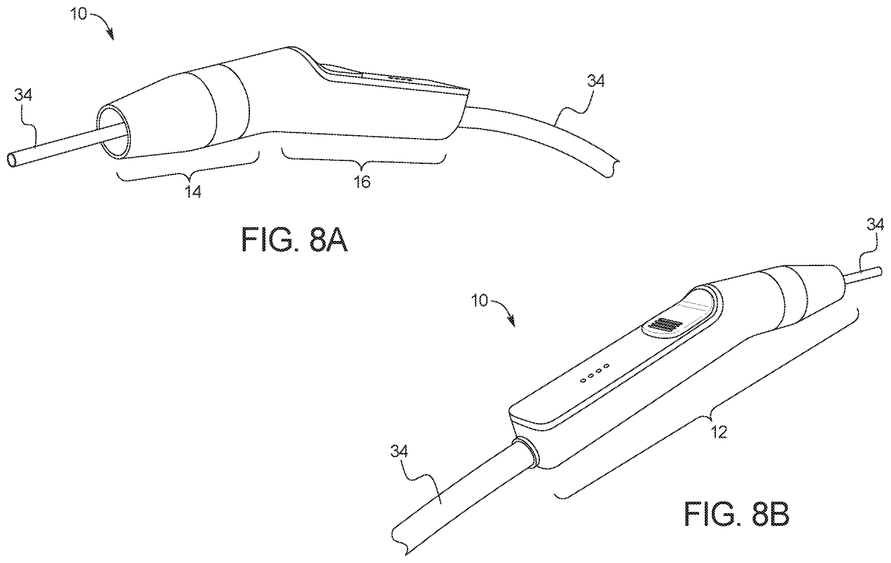

FIGS. 8A-8B illustrate a configuration of the device 10 wherein the handle portion 16 and pistol portion 14 are approximately linear, wherein a suction tube 34 can connect to an end of the handle portion 16 that can be connected to a portable pump or permanent pump.

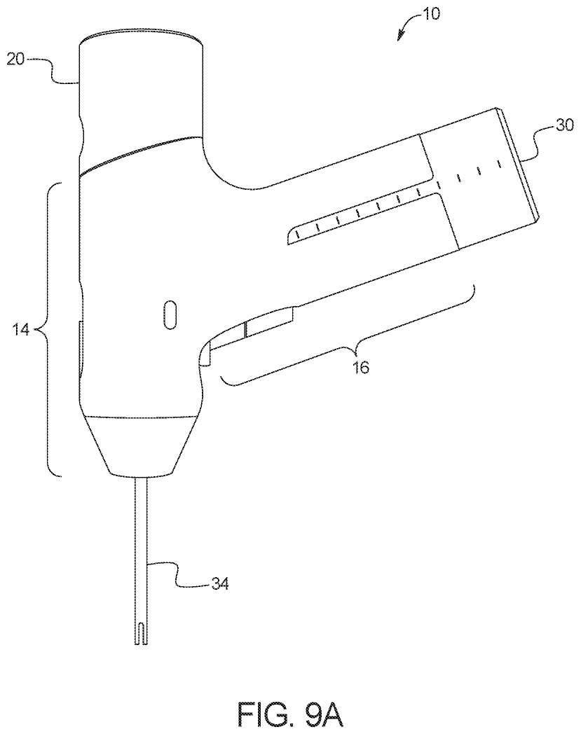

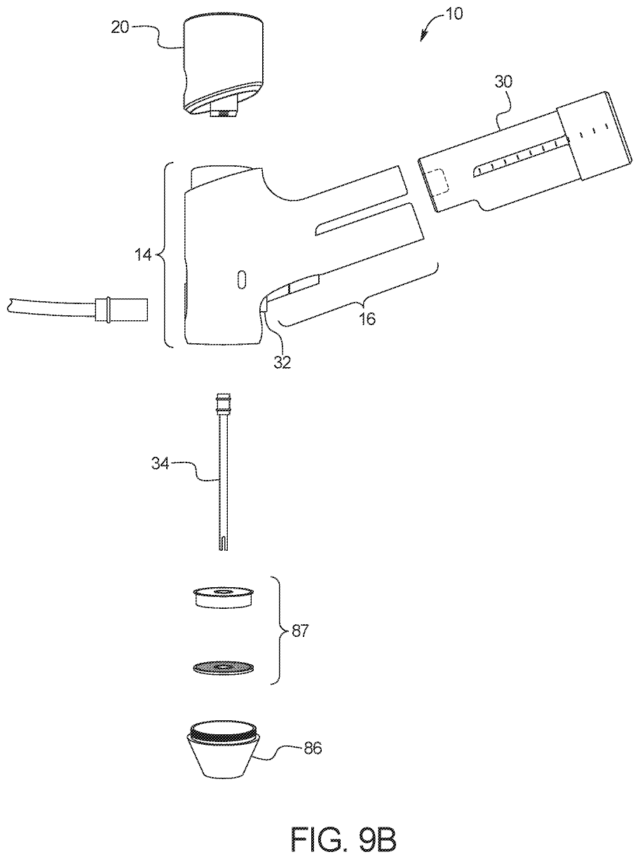

FIGS. 9A-9C illustrate perspective and exploded views of an example of the device 10 including a fenestrated handle portion 16 to allow direct visualization of the fluid canister 30. The configuration is light weight and includes a counter-balanced Li-ion battery 20 or supercapacitor. The device 10 can include a bypass valve for an in-wall-suction and/or a separate floor-mounted suction source. As shown, different size fluid canisters can be incorporated and exchanged depending on the situation. The example includes the fluid canister 30 within the handle rather than extending from the barrel. The fluid canister 30 can be released via an ambidextrous thumb magazine release mechanism. The device 10 can also use interchangeable ULPA and debris filters, quick connect interchangeable suction tips, magnetic clasp connections for canisters with a seal, an ergonomic in-line pistol grip. The device 10 can include a sliding indwelling suction tip 86 to allow for a user to change the length of the suction tube 34, wherein the suction tip 86 is attached with the connectors 87 (e.g., washer and bolt).

The handle portion 16 or pistol portion 14 can include a light source 32 (e.g., LED) to illuminate the operative field, wherein the light source 32 can be controlled via a switch and/or dial on the trigger. The device 10 can be a cordless piece with a battery pack 20 and a removable fluid canister 30.

It should be noted that various changes and modifications to the embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein.

We claim:

1. A handheld suction device comprising:
a body including a handle portion connected to a barrel portion;
a suction intake tube positioned within the barrel portion;
a pump in fluid communication with the intake tube to provide suction;
a fluid reservoir in fluid communication with the intake tube, wherein the fluid reservoir collects suctioned liquid from the intake tube; and
a control panel positioned on the handle, wherein a user can select an option on the control panel to control a power intensity of the pump.

2. The device of claim 1, wherein the fluid reservoir is positioned at a distal end of the handle portion.

3. The device of claim 1, further comprising a sliding indwelling suction tip to allow for a change the length of the suction tube, wherein the sliding indwelling suction tip is positioned to a first end of the barrel portion, wherein the sliding indwelling suction tip surrounds the suction intake tube.

4. The device of claim 1, further comprising a battery positioned at a second end of the barrel portion.

5. The device of claim 1, further comprising a turbine fan in connection with the pump to suction air.

6. The device of claim 1, wherein the control panel includes at least button to select for gas intake or liquid intake.
